(19) United States
(12) Patent Application Publication
(10) Pub. No.: US 2010/0298825 A1
(43) Pub. Date: Nov. 25, 2010

(54) Treatment System With A Pulse Forming Network For Achieving Plasma In Tissue

(75) Inventors: Roman Slizynski, Foothill Ranch, CA (US); Trcka Milan, Northridge, CA (US)

Correspondence Address: COOK ALEX LTD, SUITE 2850, 200 WEST ADAMS STREET, CHICAGO, IL, 60606, US

(73) Assignee: Cellutions, Inc., Duluth, GA (US)

(21) Appl. No.: 12/776,105

(22) Filed: May 7, 2010

Related U.S. Application Data
(60) Provisional application No. 61/176,659, filed on May 8, 2009.

(51) Int. Cl. A61B 18/14

(52) U.S. Cl. 606/41

(57) ABSTRACT

A system for providing electrical energy to tissue to treat the tissue, including a pair of output electrodes for delivering the electrical energy to the tissue, a pulse forming network for generating short high voltage pulses of electrical energy; an isolation transformer disposed between the pulse forming network and the pair of output electrodes to deliver the short high voltage pulses of electrical energy from the pulse forming network to the pair of output electrodes and to provide voltage isolation between the pulse forming network and the electrodes, and a common mode choke disposed between the isolation transformer and the pair of output electrodes to keep the pulse current flowing out from the first electrode approximately equal to the pulse current flowing back into the second electrode to substantially reduce stray or leakage currents in the tissue. The high voltage pulses of electrical energy may be about 100 to 400 nanoseconds in duration and about 10 kilovolts to about 20 kilovolts in initial peak magnitude. Related methods are also disclosed.

### CROSS-REFERENCE TO RELATED APPLICATION

This patent application claims the benefit of, and the right of priority to, U.S. provisional patent application Ser. No. 61/176,659, filed on May 8, 2009, the entire contents of which are incorporated herein by reference.

### FIELD

The present disclosure relates to apparatus for treating tissue and, more particularly, to a pulse forming network in a tissue treatment system for achieving plasma in the tissue.

### BACKGROUND

U.S. Patent Application Publication No. US 200910281540 A1, Ser. No. 12/436,659, which is incorporated by reference herein in its entirety, discloses apparatus, systems and methods for treating a human tissue condition by subjecting tissue to electrical energy. A delivery device delivers electrical energy to the tissue from a pulse generator through a multi-needle assembly. The pulse generator generates low energy, high voltage pulses of short duration.

U.S. Pat. No. 6,326,177 to Schoenbach et al., which is also incorporated by reference herein in its entirety, describes an apparatus and method for intracellular electro-manipulation using ultra short pulses.

As taught by Schoenbach et al., target cells are subjected to one or more ultra short electric field pulses. The amplitude of the individual pulses preferably does not exceed the irreversible breakdown field of the target cells. One of the advantages of using ultra short pulses is that, since the energy of the pulses is low due to the short duration of the pulses, any thermal effects on the cells are negligible. Thus, the method may be referred to as a “cold” method, without any substantial related thermal effects.

### SUMMARY

The treatment system as described herein provides electrical energy to tissue to create a plasma condition in the tissue. The system includes a pair of output electrodes for delivering the electrical energy to the tissue and a pulse forming network for generating short high voltage pulses of electrical energy. An isolation transformer disposed between the pulse forming network and the pair of output electrodes to provide voltage isolation between the pulse forming network and the pair of output electrodes. A common mode choke is disposed between the isolation transformer and the pair of output electrodes to keep the pulse current flowing out of the pair of electrodes approximately equal to the pulse current flowing back into the pair of electrodes. For example, the high voltage pulses of electrical energy may be about 100 to 400 nanoseconds in duration and about 10 kilovolts to about 20 kilovolts in magnitude.

The methods as described herein provide electrical energy to tissue to create a plasma condition in the tissue. The method includes the steps of generating short high voltage pulses of electrical energy with a pulse forming network and supplying the high voltage pulses of electrical energy to a pair of electrodes for treating the tissue. The steps also include providing voltage isolation between the pulse forming network and the pair of electrical electrodes with an isolation transformer and applying the high voltage pulses of energy to the tissue with the pair of electrodes. Lastly, the steps include using a common mode choke to keep the pulse current flowing out of the pair of electrodes approximately equal to the pulse current flowing back into the pair of electrodes.

### BRIEF DESCRIPTION OF THE DRAWINGS

The subject matter disclosed herein, together with its objects and the advantages thereof, may best be understood by reference to the following description taken in conjunction with the accompanying drawings, in which like reference numerals identify like elements in the figures, and in which:

FIG. 1A is a schematic diagram of a preferred embodiment of a tissue treatment system including a pulse forming network, in combination with an isolation transformer and a common mode choke, for delivering electrical pulses to a pair of needles in a treatment device in accordance with the present disclosure;

FIG. 1B is an elevational view of a portion of the pulse forming network shown in FIG. 1A;

FIG. 1C is an elevational view of an energy delivery device which may be used with the tissue treatment system of FIG. 1A;

FIG. 2A is a partial longitudinal cross-sectional view of the energy delivery device of FIG. 1C;

FIG. 2B is a cut-away perspective view of a dual needle adapter in sealed packaging for the energy delivery device of FIG. 1C;

FIG. 2C is an enlarged perspective view of one of the needles in the dual needle adapter of FIG. 2B illustrating a coating which is applied to a portion thereof;

FIG. 2D is a plan view of an alternate needle assembly which has more than two needles for the energy delivery device of FIG. 1C;

FIG. 3 is a diagram illustrating a Blumlein pulse generator for delivering high voltage pulses to the energy delivery device of FIG. 1C;

FIG. 4 is a diagrammatic view of a user interface for controlling the pulse generator shown in FIG. 3 in accordance with a further aspect of the subject matter disclosed herein;

FIG. 5 is a block diagram of electronic circuitry for monitoring and controlling the pulse generator shown in FIG. 3;

FIGS. 6A and 6B are partial perspective views of an energy delivery device which utilize a needle support which may be extended to protect both needles when the delivery device is not in use;

FIG. 6B is an elevational view of a separate needle support, similar to the needle support in FIGS. 6A-6B, but with a retractable separate needle support provided for each needle;

FIG. 7A is an perspective view of another embodiment of the energy delivery device illustrated in FIG. 1C;

FIG. 7B is a partial cross-sectional view of the energy delivery device shown in FIG. 7A, which illustrates another embodiment of a disposable needle assembly with the needle assembly providing protection of the dual needles when the energy delivery device is not in use; and

FIGS. 8A and 8B are partial perspective views of an energy delivery device which are similar to FIGS. 6A-6B, but which provide a retractable cylindrical sleeve for protection of the needles when the delivery device is not in use.

### DESCRIPTION OF THE PREFERRED EMBODIMENTS

It will be understood that the features and advantages of the present disclosure may be embodied in other specific forms without departing from the spirit thereof. The present examples and embodiments, therefore, are to be considered in all respects as illustrative and not restrictive, and are not to be limited to the details presented herein.

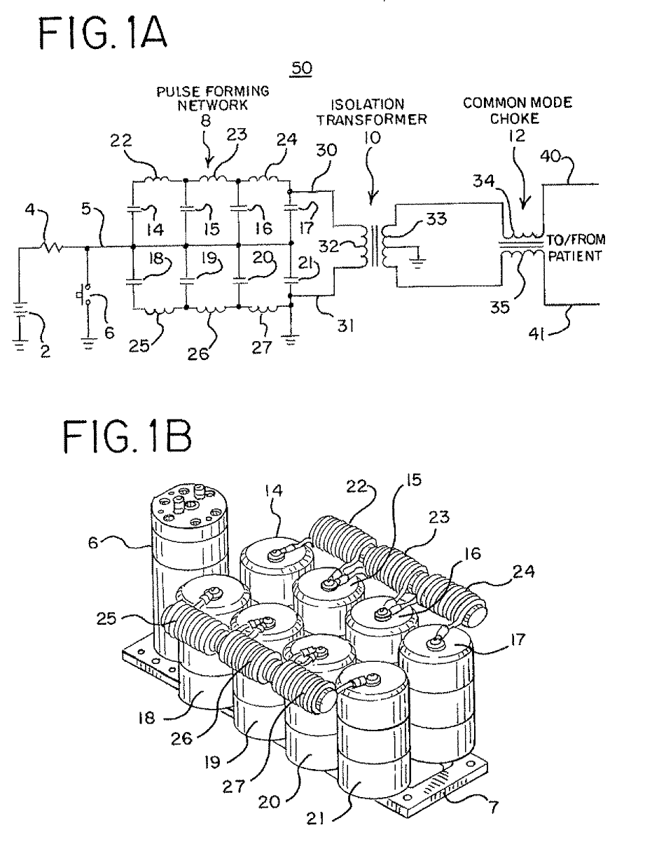

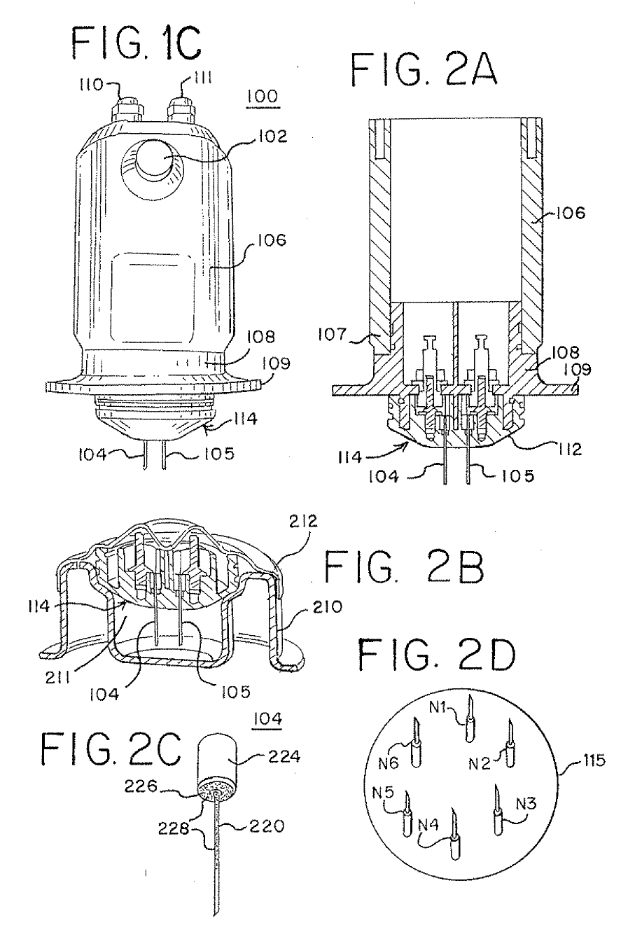

FIG. 1A illustrates an exemplary electrical circuit, generally designated 50, which may be employed in a tissue treatment system for treating tissue, such as human tissue. For example, circuit 50 may be used to generate high voltage pulses which, when applied to tissue, creates a plasma condition in the tissue, such as for destroying malignant cells or other unwanted cells.

A pulse forming network 8 consists of a plurality of capacitors 14-21 and a plurality of inductors 22-27. For example, inductors 22-27 may have an inductive value of about 3 uH, and capacitors 14-27 may have a capacitive value of about 2000 pF. Inductors 22-27, also shown in FIG. 1B, may be fabricated by hand winding about 10 to 20 turns of solid wire about a tubular form or mandrel. Due to the magnitudes of the currents generated by the pulse forming network 8, inductors 22-27 may, for example, be formed from #12 AWG wire.

Capacitors 14-21 may be of the ceramic type and also preferably have a high voltage rating such as about 40 kV. Such ceramic capacitors are commercially available, for example, from Murata Manufacturing Co. Ltd. of Japan. Capacitors 14-21 each have a terminal connected to line 5 to receive a charging current from a high voltage power source 2 through a resistor 4. Resistor 4 limits the current drawn by switch 6 when normally-open switch 6 is closed by a user to cause the pulse forming network 8 to generate high voltage pulses at its output lines 30 and 31.

Due to the high peak voltages generated by circuit 50, switch 6 may preferably be a spark gap switch. Spark gap switches are known to the prior art, such as disclosed, for example, in U.S. Pat. No. 4,897,577 to Kitzinger. As shown in FIG. 1A, output line 31 is grounded. However, if output line 30 is grounded, instead of line 31, the pulses at output lines 30-31 will be of the opposite polarity.

An isolation transformer 10 is disposed between the pulse forming network 8 and the pair of output electrodes 30-31 to deliver the short high voltage pulses of electrical energy from the pulse forming network 8 to the pair of output electrodes 40-41 and to also provide voltage isolation between the pulse forming network 8 and the output electrodes 40-41. For example, isolation transformer 10 may be formed by providing two turns for primary winding 32 on a magnetic core, such as Hitachi Metals, Ltd. of Tokyo, Japan part number FT-3KL-60450, and providing two turns for secondary winding 33 on the same core. Winding 33 preferably has a center tap for reference to ground as shown in FIG. 1A.

An embodiment of the pulse forming network 8 is shown in FIG. 1B. High voltage ceramic capacitors 14-21 have a first terminal coupled to a conductive base 7. Conductive base 7 is the electrical equivalent of line 5 in FIG. 1A. Each of the plurality of inductors 22-27 bridges a second terminal of two adjacent capacitors 14-21. The spark gap switch 6 may also be mounted to the base 7.

A common mode choke 12 is disposed between the secondary winding 33 of the isolation transformer 10 and the output electrodes 40-41. Output electrodes 40-41 may be used to apply the high voltage pulses generated by electrical circuit 50 to the tissue of a patient. For example, common mode choke 12 may be formed by providing four turns around 44 magnetic cores for primary winding 32, any by providing four turns around the same 44 magnetic cores for secondary winding 33. The magnetic cores may be the same Hitachi Metals part number FT-3KL-6045G. Other magnetic cores and other numbers of turns for primary and secondary windings 132-133 may be employed, if desired. However, the numbers of windings for the primary and secondary windings is preferably equal to provide for equal common mode currents in windings 32-33.

Since the inductors 22-27 and winding 32 of isolation transformer 10 present low D.C. impedances, the terminals of capacitors 14-21, opposite to the terminals connected to line 5, are all effectively at the ground present on line 31. Thus, all of the capacitors initially charge up from the high voltage potential present on line 5. When switch 6 is closed to generate high voltage pulses, the terminals of capacitors 14-21 attached to line 5 are suddenly taken to ground by the closure of switch 6. However, since the charge on the capacitors has not yet dissipated, the opposite terminals of the capacitors, which are connected to inductors 22-27, have a high negative voltage. Thus, the sudden change in potential at inductors 22-27 cause inductive-capacitive pulse forming network 8 to resonate at a frequency determined by the capacitive values of capacitors 14-21 and the inductance values of inductors 22-27, thereby generating high voltage pulses across lines 30-31.

These high voltage pulses at lines 30-31 are coupled to winding 32 of isolation transformer 10. Isolation transformer 10 isolates a patient from the pulse forming network 8. Isolation transformer 10 has a second winding 33, which may have a center tap connected to ground, as shown in FIG. 1A. Isolation transformer 10 may be built by using insulated high voltage wire wound on high permeability magnetic cores. With its secondary winding 33 center tapped, winding 33 provides output voltage pulses which are symmetric with respect to ground. The isolation transformer 10 also provides impedance matching of the output of pulse forming network at winding 32 with the load impedance present at winding 33.

Winding 33 of isolation transformer 10 is coupled to common mode choke 12. Common mode choke 12 has two windings. A first winding 34 is connected at one end to one end of winding 33 of the isolation transformer 10, and at its opposite end to an electrode 40. A second winding 35 of common mode choke 12 is connected at one end to a second end of winding 33 of the isolation transformer, and at its opposite end to a second electrode 41. The purpose of the common mode choke 12 is to ensure that the pulse current flowing out of electrode 40 or 41 is equal to the current flowing back in through electrode 41 or 40, respectively. Thus, stray currents or leakage currents, which may flow through the patient's body and return to the treatment system by way of stray capacitances, are substantially reduced or eliminated. It has also been observed that leakage currents are the probable cause of hard muscle contractions in test animals. Such muscle contractions were eliminated when the common mode choke was used.

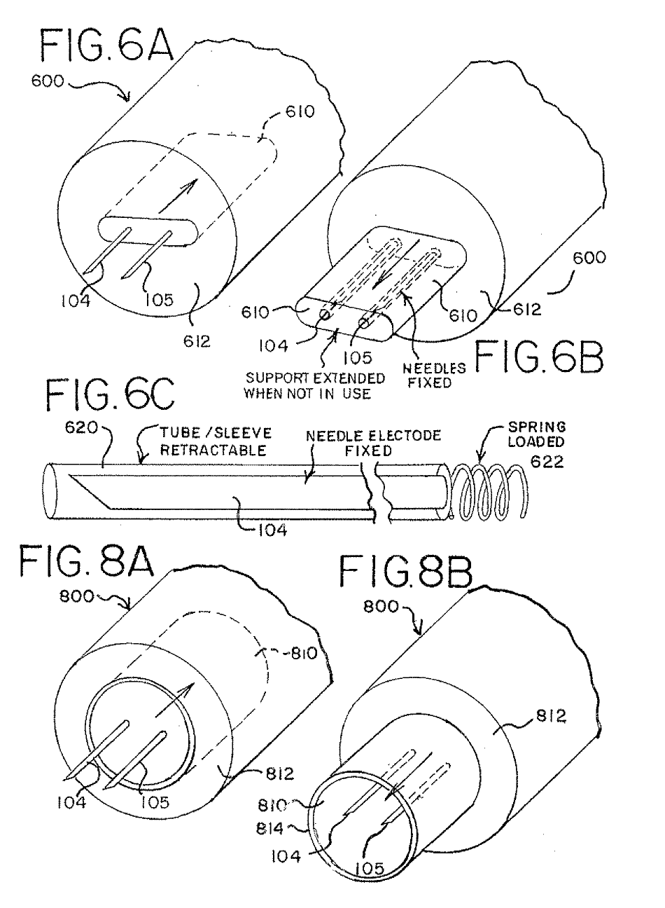

Electrodes 40-41 may be in the form of dual needles, such as dual needles 104-105 shown in FIG. 1B. These dual needles 104-105 are typically part of a treatment device 100, or part of a treatment device 600, 700 or 800 shown in FIGS. 6A-8B, respectively. When the pair of needles 40-41 (e.g., needles 104-105 in FIGS. 1B, 2A, 2B, 3 and 6A-8B) is inserted into tissue to be treated, the high voltages generated by the pulse forming network 8 are applied to the tissue to create a plasma condition in the tissue to destroy unwanted cells, such as malignant cells. High voltages are required to move electric charges through human tissue and to create high plasma currents. The value of this voltage threshold is a function of many variables, which include, but are not limited to, tissue density, blood saturation, temperature, and the distance to other tissue types. For a given distance between the needles 40-41, the threshold voltage will typically vary in thousands of volts, and may be, for example, about 15 kV.

For example, charging of capacitors 14-21 in the pulse forming network 8 to a nominal 10 kV, and using the pulse forming network 8 without any impedance matching, the output voltage at the needles 40-41 will slew to a range of about 10 kV to about 20 kV at the initiation of pulse generation. Thereafter, the peak voltages generated will quickly decay to lower peak values. If desired, the circuit 50 can be modified to accommodate other ranges of voltages.

The duration of the high voltage pulse may be in a range of about 100 nanoseconds to about 400 nanoseconds. If desired, the pulse duration may be further varied by the changes in the values of the inductors and the capacitors. Pulses of such voltage magnitude and pulse duration can typically create a plasma condition in tissue.

FIGS. 1C-8B and the corresponding discussion which follows disclose a delivery device and system of the type shown in pending U.S. Patent Application Publication No. US 2009/0281540, U.S. patent application Ser. No. 12/436,659 (the '659 application), filed on May 6, 2009, and entitled “Apparatus, Systems and Methods for Treating a Human Tissue Condition”. The improved delivery device disclosed therein delivers electrical energy from a pulse generator through a dual-needle assembly. The pulse generator generates low energy, high voltage pulses of short duration, and the pulse generator has a resistive network to limit the current flow during an energy pulse if a high conductivity condition exists.

The apparatus and methods disclosed in the '659 application may be applicable to, or usable with, the present disclosure of the circuit 50 in FIG. 1A. For example, and as noted above, the output electrodes 40-41 of circuit 50 in FIG. 1A may comprise the needles 104-105 of a delivery device 100 shown in FIG. 1C, or the variations of delivery device 100 shown in FIGS. 6A-8B.

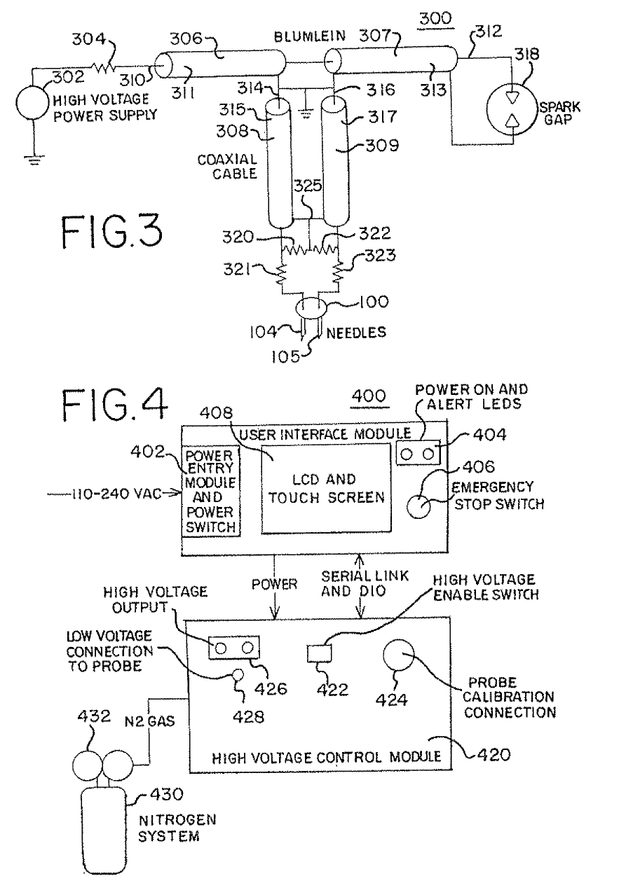

An embodiment of an electrical pulse delivery device, generally designated 100, and which may be substituted for the electrodes 40-41 of circuit 50 in FIG. 1A, is shown in FIG. 1C. Delivery device 100 provides ultra-short pulses of energy for an intracellular electro-manipulation or other treatment in accordance with the subject matter disclosed herein. A button 102 is disposed on the delivery device, such as near the top of delivery device 100. Button 102 operates as an electrical switch to provide electrical energy from a pulse generator 300 in FIG. 3 via a pair of input terminals 110-111 to a pair of needles 104 and 105 disposed on delivery device 100. For example, when button 102 is depressed, delivery device 100 provides pulses of energy from the pulse generator 300 to the pair of needles 104 and 105 for the intracellular electro-manipulation treatment. Upon release of button 102, the electrical path between the pulse generator 300 and the needles 104 and 105 is interrupted, and further treatment is automatically terminated.

A portion of delivery device 100 includes a generally cylindrical housing 106. As seen in FIG. 2A, a lower end 107 of the housing 106 is suitable for receiving an adapter 108. Adapter 108 has a radially extending flange 109 of larger diameter than housing 106, which may assist a user in holding delivery device 100 during a treatment procedure. A dual needle assembly 114 (FIG. 2A) fits onto the bottom end of adapter 108. Dual needle assembly 114 may have an exterior domed surface 112 through which the pair of needles 104 and 105 extends downwardly.

Preferably, the dual needle assembly 114 is disposable and is sealed for hygienic reasons. As shown in FIG. 2B, dual needle assembly 114 may come prepackaged. A lower package portion 210 provides a chamber 211 for protecting needles 104-105 prior to use, and an upper package portion 212 seals to lower package portion 210. Since needles 104 and 105 are intended to be electrically conductive to supply electrical energy to tissue to be treated, most of the remainder of assembly 114 is preferably constructed of an insulative material, such as an ABS (acrylonitrile butatiene styrene) plastic. Side portions of assembly 114 may provide a frictional fit to retain the assembly 114 onto the lower end of the adapter 108. Alternatively, assembly 114 may be threaded to secure assembly 114 to adapter 108.

Needles 104 and 105 are preferably micro-needles, which may be made, for example, from solid 30 gauge stainless steel (316) stock. The tips of needles 104 and 105 may be hypodermic-style. That is, the tips may be formed with cutting edges to facilitate relatively painless and easy penetration of the skin. FIG. 2C illustrates one of the needles 104. As illustrated in FIG. 2C, a coating 228 is preferably applied to a proximal end 220 of needle 104, with the distal end 222 uncoated. An underside 226 of the head 224 of needle 104 may also have the coating 228 applied thereto.

The purpose of coating 228 at the upper end 220 of needle 104 is to avoid application of stronger electrical fields by delivery device 100 to dermal tissues while the lower uncoated end 222 is applying electrical fields to sub dermal tissue, such as fat cells and connective tissue called septae. Coating 228 is preferably relatively uniform in thickness and without any voids, such as pinholes. For example, coating 228 may be a parylene coating, which is deposited by a vapor-phase deposition polymerization process. Parylene has a low coefficient of friction, very low permeability to moisture and a high dielectric strength. Other examples for the coating 228 include polyimide, polyester, diamond, Teflon and siloxane. While needle 104 is shown in FIG. 2C and described above, it will be appreciated that needle 105 is similar to needle 104, including the coating 228. For hygienic reasons, the entire micro needle assembly 114, including needles 104 and 105, may be disposable.

For example, the needles 104 and 105 may extend about 5 mm to 15 mm, and, typically about 8 mm, from the bottom surface 112 of delivery device 100, with the proximal 3 mm to 8 mm of needles 104 and 105 having the insulating parylene coating 228. The parylene coating 228 is intended to extend through the dermis during a treatment procedure, thus protecting the dermis by substantially reducing the electrical field between needles 104 and 105 in the vicinity of the dermis. By way of example, the dual-needle delivery device 100 discussed herein may subject the target cells to a pulse in the range of 10 nanoseconds to 500 nanoseconds (10×10⁻⁹ seconds to 500×10⁻⁹ seconds) having an average electric field strength (“E”) of about 10 kV/cm to 50 kV/cm, and, typically of about 30 kV/cm, at a pulse rate of about 1 to 10 pulses per second.

With reference to FIG. 2A, the apparatus and system may also include one or more contact switches 116-118 at the distal face 114 of the delivery device 100 in contact with skin. A necessary condition for delivery of the electrical pulse can be activation of the contact switches when skin is pressed against the distal face 114, including one or any combination of the contact switches 116-118. This ensures that there is no significant air gap between the face 114 of the delivery device 100 and the skin, and consequently, the likelihood of energy delivery occurring on top of the skin surface is reduced or eliminated.

An alternate multiple needle array 115, which provides more than two needles 104-105 in the dual needle assembly 114, is shown in FIG. 2D. In the example of FIG. 2D, the multiple needle array 115 provides six needles N1 through N6. These needles may be partially insulated, as with needles 104-105. By way of example, voltage can be first applied between needles N1 and N2, then between needles N1 and N3, and so on. For N needles, the distinct number of pairs is (N*N−(N(N(N+1)/2))=36−21=15. These 15 pairs are N1-N2, N1-N3, N1-N4, N1-N5, N1-N6, N2-N3, N2-N4, N2-N5, N2-N6, N3-N4, N3-N5, N3-N6, N4-N5, N4-N6 and N5-N6. Voltage can be applied to all of these distinct pairs, or to some of these distinct pairs. Other configurations and choices of pairs are also contemplated.

As described above, the system delivers very short pulses of low energy to the tissue being treated. The schematic diagram in FIG. 3 illustrates a pulse generator, generally designated 300, of the Blumlein transmission line type, for generating low energy/high voltage pulses of short duration. In this embodiment, the ultra-short pulses are generated by pulse generator 300, but such pulses could also be generated using a pulse-forming network or by any other suitable methods. Pulse generator 300 generally consists of a high voltage power supply 302, four sections of coaxial cable 306-309 and a spark gap 318. A resistor 304 may be disposed between the high voltage power supply and the first coaxial section 306.

Inner conductors 310 and 312 of coaxial sections 306 and 307 connect to one of the leads of the spark gap 318. The other lead of spark gap 318 connects to the outer sheath 313 of coaxial section 307. Near coaxial sections 308 and 309, the outer sheaths 311 and 313 of coaxial sections 306 and 307 are grounded, as well as the inner conductors 314 and 316 of coaxial sections 308 and 309. At the opposite ends of coaxial sections 308 and 309, the outer sheaths 315 and 317 are connected together at a node 325. Inner conductor 314 of coaxial section 308 is connected to a pair of resistors 320 and 321, and inner conductor 316 of coaxial section 309 is similarly connected to another pair of resistors 322 and 323. Opposite ends of resistors 320 and 322 are connected to node 325. Opposite ends of resistors 321 and 323 are connected to needles 104 and 105, respectively. Collectively, resistors 320-323 form a balanced resistor network at the output of pulse generator 300.

The spark gap 318 may be filled with nitrogen or any other suitable gas. The internal pressure of the nitrogen in the spark gap may be regulated to control the voltage at which the spark gap breaks down, thereby also controlling the amount of energy delivered to the needles 104 and 105 by the pulse generator 300. When the spark gap breaks down, a high voltage, short duration pulse will be delivered to the needles through the balanced resistor network consisting of resistors 320-323. In an embodiment, all of resistors 320-323 may be about 50 ohms. The magnitude of the voltage delivered to the patient is determined by the spark gap 318. The spark gap will breakdown when the voltage across its electrodes exceeds the dielectric strength of the gas in the spark gap. The dielectric strength of the gas is controlled by the gaseous pressure within the spark gap. Thus, controlling the gaseous pressure also controls the magnitude of the voltage delivered.

In order to safely and reliably deliver short high-voltage pulses to a patient during a treatment procedure, adequate controls and monitors are required. The subject matter disclosed herein is also concerned with such controls and monitors. The first set of controls relate to ensuring that the voltage delivered to the patient is correct and accurate. The voltage delivered to the patient is selected by the operator through a user interface module, generally designated 400 in FIG. 4. Module 400 may include a power entry module with a power switch 402, indicators 404 for power on and alerts, such as light emitting diodes (LEDs), an emergency stop switch 406 and a touch sensitive screen 408 for displaying and selecting operating modes, menus of available options, and the like.

Associated with user interface module 400 is a high voltage control module 420. Module 420 may include a high voltage enable switch 422, a probe (also referred to herein as delivery device 100) calibration connection 424, a high voltage output 426 for supplying the high voltage pulses to delivery device 100, and a low voltage connection 428 for the delivery device 100. A regulator 432 monitors and supplies nitrogen gas to spark gap 318 from a source of compressed nitrogen 430.

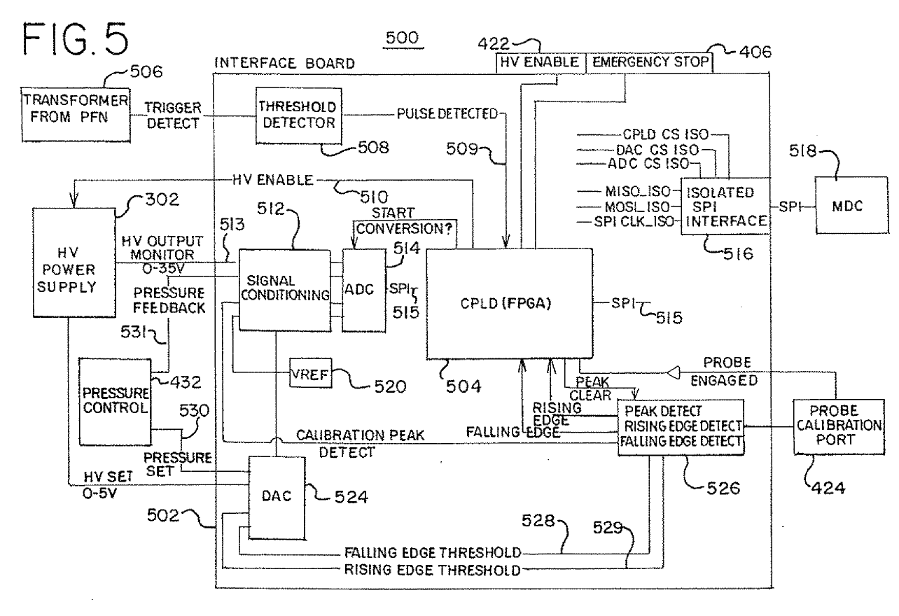

FIG. 5 illustrates, in block diagram format, the electronic circuitry, generally designated 500, which may be contained within the high voltage control module 420 shown in FIG. 4. Much of circuitry 500 may be on a interface circuit board 502. Circuitry 500 is monitored and controlled by a complex programmable logic device (CPLD) 504. Alternatively, CPLD 500 may be a field-programmable gate array (FPGA) or any suitable microprocessor or microcontroller. The high voltage (HV) pulses generated by pulse generator 300 and supplied to delivery device 100 may be monitored in any of a variety of ways. For example, the HV pulses may be monitored by sensing the voltage across one of the resistors 321 or 323 in FIG. 3. A resistor divider (not shown) may be connected across resistor 321 to reduce the high voltage pulse to a lower level more suitable for the electronic circuitry 500. A pulse transformer 506 may be used to supply the pulse to circuitry 500, while also providing DC isolation between the circuitry and the pulse generator. A threshold detector 508 receives pulse signals from transformer 506 and provides pulse detection information to CPLD 504 via line 509 if any pulse exceeds a predetermined threshold.

CPLD 504 enables the HV power supply 302 via line 510. Signal conditioning circuitry 512 monitors the output voltage of the HV power supply on line 513. In this respect, signal conditioning circuitry 512 may have a voltage reference for comparison purposes. An analog to digital converter (ADC) 514 supplies the monitored information to CPLD 504 via a serial peripheral interface (SPI) bus. The SPI bus is also routed to other portions of the circuitry 500, such as to an isolated SPI interface 516 which may supply information to external sources, such as a master data controller 518.

Digital information concerning falling edge threshold and rising edge threshold is provided from peak detector 526, via lines 528 and 529, to a digital to analog converter (DAC) 524. DAC 524 then provides a pressure set signal on line 530 to pressure control 432 to regulate the pressure of nitrogen in the spark gap 318. As previously explained, control of the pressure in spark gap 318 controls the magnitude of the high voltage pulses generated by pulse generator 300. Pressure feedback information is provided from pressure control 432 on line 531 to the signal conditioning and thence to ADC where it is sent via the SPI bus to CPLD 504.

The CPLD or microprocessor 504 controls the gas pressure regulator 432 in setting and monitoring the gaseous pressure within the spark gap 318. The microprocessor also monitors the voltages going to the Blumlein pulse generator 300 and the voltage across the load resistors 320-323 on the output of the pulse generator using resistor dividers, pulse transformer 506 and analog to digital converter 514. Prior to use on the patient, the delivered voltage at the needles 104-105 is adjusted to ensure a proper value. This process starts by setting the spark gap pressure to an empirically generated first guess estimated to give the proper voltage. The Blumlein pulse generator 300 is fired and the pulse generator voltages are monitored. The pressure is then adjusted based on the difference between the measured output voltage and the desired output voltage. The adjustment process continues until the difference between the measured and desired is within an acceptable level.

The adjustment is preferably proportional control. However, the adjustment could also include differential and integral control. The control can be based on either the monitored pulse generator input or output signal. Using the pulse generator input signal requires monitoring the input voltage and holding the peak value from the time that the high voltage power supply (HVPS) 302 is activated until the pulse is delivered at the needles 104-105. Delivery of the pulse can be detected by either sensing a rapid decrease in the pulse generator input, a pulse on the pulse generator output or an optical signal from the spark gap. Using the pulse generator output signal may require detecting the rising and falling edges of the pulse and averaging the values between these two edges.

An alternate method for monitoring the voltage is to implement a calibration port 424 on the system. This calibration port 424 allows the distal end of the delivery device 100 to be connected to the console 420. The distal electrode voltage is then monitored and the spark gap pressure is controlled to ensure that the distal electrode voltage matches the desired output voltage within appropriate limits. This method will compensate for any losses or changes to the voltage induced by the patient cable and/or the delivery device.

A second set of controls is related to controlling the pulse delivery rate. The control of the pulse delivery rate is selected by the operator through the user interface 400. The microprocessor 504 controls the delivery of each pulse by commanding the HVPS 302 to go to a predetermined high voltage level that is selected to be higher than the desired voltage delivered to the patient. In this embodiment, the microprocessor controls the HVPS command through a field programmable gate array (FPGA) 504. This FPGA buffers the command to the HVPS 302 and controls the slope of the command to mitigate against excessive overshoot of the HVPS output. The output of the HVPS is feed into the pulse generator 300 through a series resistor and appropriate protection diodes. The microprocessor 504 will initiate these pulses at the rate determined by the user interface 400, such as by selection on screen 408. Several monitors ensure that the pulses delivered are within predetermined parameters. If any of these monitors indicate that the pulse has not been delivered, microprocessor 504 will inhibit any new pulses from being initiated and will alert the operator to the problem.

One risk for any high voltage delivery system is that some other component in the system breaks down at a lower voltage than the spark gap 318. If this occurs, no pulse, an improperly shaped pulse or a lower voltage pulse could be delivered to the patient. If any failures within the system are detected or if delivered pulses are not within established parameters, subsequent delivery of pulses will be terminated and the operator will be alerted.

In accordance with another aspect, the subject matter disclosed herein may be used by a physician to treat cellulite by inducing selective adipocyte death in the subcutaneous fat layer (SFL), or cutting of collageneous septae, or both, such as by plasma spark discharge. Adipocyte death may be caused by apoptosis or necrosis, both considered cell lysis. The dead adipocytes will be naturally reabsorbed by the body. Fewer adipocytes in the SFL will reduce the pressure on the dermis, blood vessels and lymphatic system in the affected area, which will typically lead to an improved cosmetic experience. The subject matter disclosed herein may also have an effect of cutting or ablating or denaturing septae that tether the dermis to the underlying fascia. These effects on the septae will lead to improvement in the appearance of cellulite dimples, for example, by releasing the tension on the dermis.

In accordance with a further aspect of the subject matter disclosed herein, needles 104-105 may be force assisted for insertion into the skin. One of the problems associated with small gauge needles, such as about 30 gauge needles, is that they tend to bend while insertion into the skin if the needles are not substantially perpendicular to the skin during insertion. Thus, care must be taken while inserting the needles into the skin to apply forces perpendicular to the skin surface, and in the direction of the needles, to avoid bending the needles. Thus, in accordance with another aspect of the subject matter disclosed herein, the needles 104-105 may be retractable into the delivery device 100. Upon actuation, the needles 104-105 are quickly forced or shot out to their full distal position, as illustrated in FIG. 1C. The needles 104-105 are then held in this distal position by mechanical means or by application of force from the power source while therapeutic electrical pulses are delivered through the needles to the patient. Following the electrical pulse treatment, the needles may again be retracted into the delivery device 100.

In accordance with yet another aspect of the subject matter disclosed herein, an energy delivery device 600 may be provided with a retractable needle support 610 or 620, as illustrated by the embodiments shown in FIGS. 6A, 6B and 6C. In accordance with this aspect of the subject matter disclosed herein, delivery device 600 and needles 104-105 are provided with a retractable needle support 610 which surrounds the needles 104-105 and which extends out of the bottom surface 612 of the delivery device 600 as shown in FIG. 6B. Upon insertion of the needles 104-105 into the skin of a patient, the retractable support 610 comes into contact with the skin of the patient and the retractable support 610 is pushed back into the interior of the delivery device 600 as shown in FIG. 6A, thereby permitting the ends of the needles to penetrate the skin for the electric pulse treatment of the tissue. The retractable support 610 thus holds the needles 104-105 in position during insertion and assists in preventing bending of the needles during insertion.

A desirable characteristic of the retractable support 610 is to house the needles 104-105 in a manner which protects the needles from bending or from encountering other damage when not in use. For example, the retractable support 610 may be a tube-like structure of a length sufficient to cover the ends of the needles 104-105, with internal diameters sufficiently large to accommodate the smaller diameter needles, but also of sufficiently small diameter to prevent any significant bending of the needles 104-105 during insertion. Retractable support 610 may be of any suitable shape, such as of a modified oval cross-sectional shape shown in FIGS. 6A and 6B, cylindrical cross-sectional shape, square, rectangular, or other cross-sectional shapes.

Alternatively, a separate retractable support 620 in FIG. 6C may be used about each needle 104 or 105. Retractable support 602 may be of any suitable shape, such as the cylindrical cross-sectional shape illustrated in FIG. 6C. In a manner similar to retractable support 610, each of retractable supports 620 may be pushed back into the interior of the delivery device 600 as the retractable supports come into contact with the skin, thereby permitting the ends of the needles to penetrate the skin for the electric pulse treatment of the tissue.

Either of the retractable supports 610 or 620 may be biased by light pressure supplied, such as by a spring 622 shown in FIG. 6C to extend the supports about the ends of the needles 104-105 when not in use, to retract into the delivery device 600 when in use, and to again extend about the ends of the needles when the treatment is completed. Such a retractable support will also protect the needles from bending or other damage when not in use and may also protect the physician or staff from injury when not in use.

In accordance with another aspect of the subject matter disclosed herein, the delivery device 100 may utilize vacuum-assisted skin engagement. Current and prior art procedures require the physician to hold a delivery device perpendicular to the skin with moderate pressure. If the orientation of the delivery device changes, or if the pressure of the delivery device 100 against the surface of the skin changes, the electrical conditions between the adipose tissue, the pulse generator 300 and the two needles 104-105 may change, resulting in a higher than desired current level. Additionally, air may become entrapped between the needles which may provide a leakage current path.

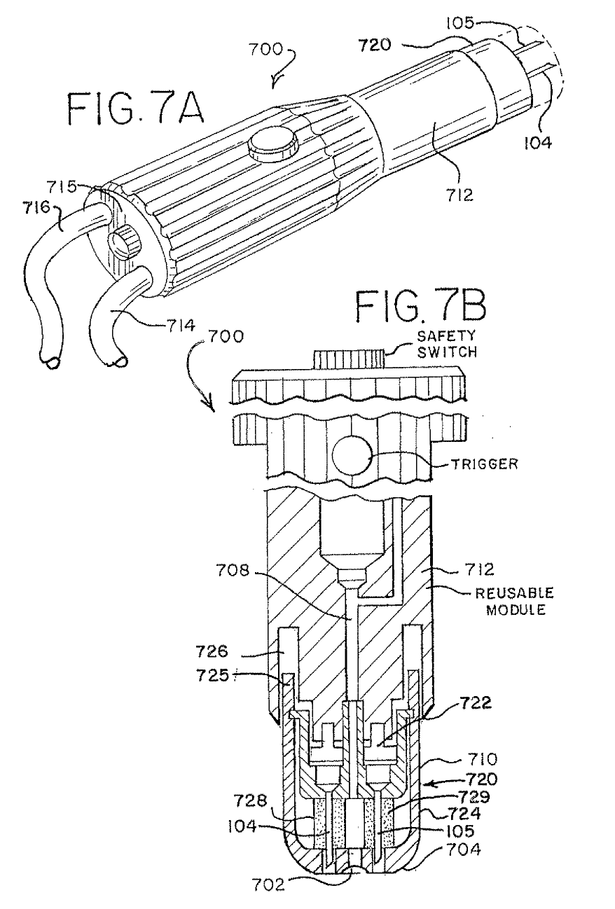

Illustrated in FIGS. 7A and 7B is a delivery device 700, which may use a light vacuum to assist in pulling the surface of the skin into contact with the bottom surface 704 of the delivery device. Further, once the bottom surface 704 of the delivery device 700 is in engagement with the skin of the patient, the light vacuum assists in retaining the bottom surface of the delivery device in contact with the skin. Thus, any effects due to movement of the patient or the physician are minimized as the patient's skin tends to move with any corresponding movement of the delivery device. For example, the vacuum may be supplied via an orifice 702 in the distal or bottom face 704, such as between needles 104 and 105. Orifice 702 is in the reusable module portion 712 of device 700 which is also in vacuum communication with an internal vacuum passageway 708 in the disposable module portion 710 of device 700. As shown in FIG. 7B, the portion of orifice 702 which meets the bottom surface 704 of the disposable module 710 may be enlarged for application of the vacuum thereat to a correspondingly larger area of the skin. A goal of using a vacuum is to ensure good contact of the delivery device 100 with the skin.

Another embodiment of a disposable needle assembly 720 is shown in FIG. 7B for use with energy delivery device 700. Needles 104-105 electrically connect to delivery device 700, such as by a mini banana plug interface 722, to receive high voltage pulses which are provided by one of the electrical lines 714 or 716 (FIG. 7A) connected to the back end 715 of device 700. The other line 716 or 714 may be used for control signals. Needle assembly 720 includes an outer sleeve 724. The upper end 725 of outer sleeve 724 fits partially into an annular recess 726 defined in the front end 712 of device 700. A ring 728 and 729 of closed cell foam is internally disposed about each needle 104 and 105, respectively. These foam rings 728-729 tend to bias the outer sleeve 724 to the position shown in FIG. 7B where the needles 104-105 are not exposed, but are substantially within outer sleeve 724.

However, when the bottom face 704 of the outer sleeve 724 is applied against the skin of a patient, the foam rings 728-729 are compressed such that needles 104-105 penetrate the skin. At the same time, the upper end 725 of outer sleeve 724 moves upwardly within the annular recess 726. If desired, the limit of needle penetration in the skin can be provided when the upper end 725 contacts the end of the annular groove 726, or when the foam rings 728-729 are fully compressed. The foam rings may be of a foam material which has memory to return to its uncompressed state when a treatment is completed. For example, foam rings 728-729 may be made of a closed cell foam material.

Another embodiment for protecting for the needles 104-105 is shown in FIGS. 8A and 8B. In this embodiment, a sleeve 810 may be retracted for treatment of a patient and the sleeve 810 may be extended when the delivery device 800 is not in use. For example, sleeve 810 may be biased to the extended position shown in FIG. 8B by a spring or the like, in a similar manner to spring 622 in FIG. 6C. Sleeve 810 may be cylindrical in cross-section shape, or oval or other shapes. When sleeve 810 is fully extended, as shown in FIG. 8B, a front edge 814 of sleeve 810 extends forwardly of the tips of needles 104-105. The embodiment shown in FIGS. 8A-8B has some advantages when delivery device uses vacuum assisted treatment. For example, when delivery device 800 is provided with a vacuum orifice, such as orifice 702 shown in FIG. 7B, the entire area within sleeve 810 will be under vacuum as soon as the front edge 814 of sleeve 810 comes into contact with the skin. This will assist in pulling the skin into contact with the needles 104-105 and will also help prevent lateral movement of the delivery device 800 thereby preventing bending of needles 104-105 during insertion.

While particular embodiments have been shown and described, it will be obvious to those skilled in the art that changes and modifications may be made therein without departing from the spirit of the claims in their broader aspects.

1. A system for providing electrical energy to tissue to treat the tissue, said apparatus comprising:
a pair of output electrodes, including a first electrode and a second electrode, for delivering the electrical energy to the tissue;
a pulse forming network for generating short high voltage pulses of electrical energy; and
an isolation transformer disposed between the pulse forming network and the pair of output electrodes to deliver the short high voltage pulses of electrical energy from the pulse forming network to the pair of output electrodes and to provide voltage isolation between the pulse forming network and the electrodes.

2. A system for providing electrical energy to tissue to treat the tissue in accordance with claim 1, said apparatus further comprising:
a common mode choke disposed between the isolation transformer and the pair of output electrodes to keep the pulse current flowing out from the first electrode approximately equal to the pulse current flowing back into the second electrode thereby substantially reducing stray or leakage currents in the tissue.

3. A system for providing electrical energy to tissue to treat the tissue in accordance with claim 2, wherein said short high voltage pulses of electrical energy may be in a range of about 100 nanoseconds to about 400 nanoseconds in duration.

4. A system for providing electrical energy to tissue to treat the tissue in accordance with claim 2, wherein the duration of said short high voltage pulses of electrical energy initially reach a peak level of about 10 kilovolts to about 20 kilovolts.

5. A system for providing electrical energy to tissue to treat the tissue in accordance with claim 2, said pulse forming network further comprising:
a plurality of capacitors and a plurality of inductors arranged in an electrically resonant circuit; and
a switch element which is operable to interrupt a charging current to the plurality of capacitors.

6. A system for providing electrical energy to tissue to treat the tissue in accordance with claim 2, wherein said common mode choke comprises at least one magnetic core with a primary winding and a secondary winding wound about said magnetic core, said primary winding and said secondary winding having an equal number of turns.

7. A system for providing electrical energy to tissue to treat the tissue in accordance with claim 1, wherein said isolation transformer comprises a magnetic core, a primary winding and a secondary winding, said primary and said secondary winding wound about the magnetic core.

8. A system for providing electrical energy to tissue to treat the tissue in accordance with claim 7, wherein said secondary winding has a center tap for reference to a ground potential.

9. A system for providing electrical energy to tissue to treat the tissue in accordance with claim 1, further comprising:
an energy delivery device, said energy delivery device including the pair of output electrodes to deliver the electrical energy generated by the pulse forming network to the tissue.

10. A system for providing electrical energy to tissue to treat the tissue in accordance with claim 9, wherein said pair of output electrodes comprises a pair of needles.

11. A method for providing electrical energy to tissue to treat the tissue, said method comprising the steps of:
generating short high voltage pulses of electrical energy with a pulse forming network;
supplying the high voltage pulses of electrical energy to a pair of electrodes for treating the tissue;
providing voltage isolation between the pulse forming network and the pair of electrodes with an isolation transformer, thereby isolating the patient from the pulse forming circuit;
applying the high voltage pulses of energy to said tissue with said pair of electrodes to create a plasma current in the tissue; and
equalizing current flowing out of, and in to, the tissue to assure minimum leakage current for patient safety.

12. A method for providing electrical energy to tissue to treat the tissue in accordance with claim 11, said method comprising the further step of:
using a common mode choke to keep the pulse current flowing out of the pair of electrodes approximately equal to the pulse current flowing back into the pair of electrodes to substantially reduce stray or leakage currents in the tissue.

13. A method for providing electrical energy to tissue to treat the tissue in accordance with claim 11, wherein the duration of said short high voltage pulses of electrical energy may be in a range of about 100 nanoseconds to about 400 nanoseconds in duration.

14. A method for providing electrical energy to tissue to treat the tissue in accordance with claim 11, wherein said short high voltage pulses of electrical energy initially reach a peak level of about 10 kilovolts to about 20 kilovolts.

15. A method for providing electrical energy to tissue to treat the tissue in accordance with claim 11, said method comprising the further step of:
providing said pulse forming network by arranging a plurality of capacitors and a plurality of inductors in an electrically resonant circuit; and
providing a switch element which is operable to interrupt a charging current to the capacitors.

16. A method for providing electrical energy to tissue to treat the tissue in accordance with claim 12, wherein said common mode choke comprises at least one magnetic core with a primary winding and a secondary winding wound about said magnetic core, said primary winding and said secondary winding having an equal number of turns.

17. A method for providing electrical energy to tissue to treat the tissue in accordance with claim 11, wherein said isolation transformer comprises a magnetic core, a primary winding and a secondary winding, said primary and said secondary winding wound about the magnetic core.

18. A method for providing electrical energy to tissue to treat the tissue in accordance with claim 17, wherein said secondary winding has a center tap for reference to a ground potential.

19. A method for providing electrical energy to tissue to treat the tissue in accordance with claim 11, said method comprising the further step of:
providing an energy delivery device, said energy delivery device including the pair of output electrodes to deliver the electrical energy generated by the pulse forming network to the tissue.

20. A method for providing electrical energy to tissue to treat the tissue in accordance with claim 19, wherein said pair of output electrodes comprises a pair of needles.
